(19) United States
(12) Patent Application Publication
(10) Pub. No.: US 2012/0277532 A1
(43) Pub. Date: Nov. 1, 2012

(54) ENDOSCOPE GAS-SUPPLY SYSTEM

(75) Inventor: Nobuyuki TORISAWA, Ashigarakami-gun (JP)

(73) Assignee: Fujifilm Corporation, Tokyo (JP)

(21) Appl. No.: 13/452,796

(22) Filed: Apr. 20, 2012

(30) Foreign Application Priority Data
Apr. 28, 2011 (JP) JP2011-101673
Apr. 28, 2011 (JP) JP2011-101674

(51) Int. Cl. A61B 1/015

(52) U.S. Cl. 600/118

(57) ABSTRACT

Provided is an endoscope gas-supply system which enables to measure the intraluminal pressure of a human lumen unaffectedly by body motions, and to stably perform automatic gas supply into the lumen. In the endoscope gas-supply system, a gas-supply duct for automatic gas supply from a gas-supply apparatus into the lumen includes a buffer tank which has a predetermined volume. The intraluminal pressure of the lumen is indirectly obtained from a result of measurement of a pressure sensor in the gas-supply apparatus, and pressure control is performed such that the intraluminal pressure of the lumen becomes a set value. At this time, intraluminal pressure fluctuation due to volume variation of the lumen is absorbed at the buffer tank, so the pressure sensor can perform pressure measurement stably, and stabilization of automatic gas supply into the lumen can be achieved.

### BACKGROUND OF THE INVENTION

1. Field of the Invention

The present invention relates to an endoscope gas-supply system, and in particular to an endoscope gas-supply system to supply predetermined gas through a gas-supply tube into a lumen of a subject.

2. Description of the Related Art

In recent years, a technique to observe and treat a site intended to be treated in a human lumen (for example, the stomach, large intestine, esophagus, or the like) with an insertion unit of an endoscope inserted in the lumen has been performed. At this time, for the purpose of securing a region for operating a treatment instrument while securing the field of view of the endoscope, the lumen is inflated by injecting gas into the lumen through a gas-supply tube of the endoscope (for example, see Japanese Patent Application Laid-Open No. 2006-288881). At this time, it is preferred that, as the gas injected in the lumen, carbon dioxide gas bioabsorbed quickly is used in order to relieve patient's pain. This makes it possible to perform various treatments while checking a treatment instrument inserted through a treatment instrument channel of the endoscope while observing the site intended to be treated in the lumen.

In a conventional endoscope gas-supply system, however, the gas injection into the lumen is performed typically by manual operation of an operator, so that frequent operation is required in order to keep the luminal pressure constant. Therefore, there is the problem that the operation burden on the operator is great.

On the other hand, in a laparoscopic surgical operation, in order to secure the field of view of the endoscope or a treatment space, a gas-supply apparatus is used which performs automatic gas supply to the abdominal cavity while performing pressure control such that the intra-abdominal pressure is kept constant, while measuring the intra-abdominal pressure from a pneumoperitoneum apparatus through an insertion instrument for pneumoperitoneum, such as a pneumoperitoneum needle or a trocar, inserted in the abdomen of a patient (for example, see Japanese Patent Application Laid-Open No. 2003-250886).

### SUMMARY OF THE INVENTION

A luminal volume is much smaller than an abdominal volume (about three liters), for example, the esophagus is 80 cc in volume and the stomach is 1500 cc in volume, and the percentage of luminal volume variation due to body motions caused by breathing and the like is very high. Therefore, if the gas-supply apparatus used in the laparoscopic surgical operation is used as it is, stable pressure measurement cannot be made because of a significant negative effect from the luminal volume variation, which results in the problem that the behavior of the system easily becomes unstable.

The present invention has been made in these circumstances, and an object thereof is to provide an endoscope gas-supply system that is not affected by body motions when measuring the intraluminal pressure of a lumen so that stabilization of automatic gas supply into the lumen can be achieved.

In order to achieve the above object, an endoscope gas-supply system according to a first aspect of the present invention is endoscope gas-supply system which supplies gas from a gas supply source into a lumen of a living body, the endoscope gas-supply system comprising: a gas-supply duct which supplies gas from the gas supply source into the lumen of the living body; a pressure regulation device which regulates a pressure of the gas supplied from the gas supply source; a pressure measurement device which measures an intraluminal pressure of the lumen through the gas-supply duct; a setting device which sets a set value of the intraluminal pressure of the lumen; a pressure control device which controls the pressure of the gas regulated by the pressure regulation device based on a result of the measurement by the pressure measurement device such that the intraluminal pressure of the lumen becomes the set value set by the setting device; and a buffer tank which is provided in the gas-supply duct and which temporarily stores the gas flowing through the gas-supply duct.

According to the first aspect, the intraluminal pressure fluctuation due to volume variation of the lumen can be absorbed effectively by the buffer tank, and the pressure measurement device can measure the intraluminal pressure of the lumen stably and reliably through the gas-supply duct. This makes it possible to supply the gas regulated to a predetermined pressure stably into the lumen, thereby reducing the operation burden on the operator.

An endoscope gas-supply system according to a second aspect of the present invention is the endoscope gas-supply system according to the first aspect of the present invention, wherein the pressure regulation device is provided in the gas-supply duct.

According to the second aspect, it becomes possible to regulate the pressure of the gas supplied from the gas supply source through the gas-supply duct.

An endoscope gas-supply system according to a third aspect of the present invention is the endoscope gas-supply system according to the first or second aspect of the present invention, wherein the buffer tank is larger in volume than the lumen.

According to the third aspect, it becomes possible to improve the effect of the buffer tank to absorb the intraluminal pressure fluctuation due to volume variation of the lumen.

An endoscope gas-supply system according to a fourth aspect of the present invention is the endoscope gas-supply system according to any one of the first to third aspects of the present invention, wherein the buffer tank is a variable volume buffer tank which increases and decreases in volume according to the volume of the lumen.

According to the fourth aspect, since the volume of the buffer tank increases or decreases according to the volume of the lumen, it become possible to optimize control of the pressure of the gas supplied into the lumen.

An endoscope gas-supply system according to a fifth aspect of the present invention is the endoscope gas-supply system according to any one of the first to fourth aspects of the present invention, wherein the buffer tank has a gas-liquid separation structure which separates gas and liquid flowing through the gas-supply duct from each other.

According to the fifth aspect, since luminal contents (for example, filth, residues, or the like) flowing back through the gas-supply duct are trapped in the buffer tank, it becomes possible to prevent the luminal contents from entering an upstream side (a gas supply source side) of the gas-supply duct beyond the buffer tank. This makes it possible to perform stable gas supply into the lumen.

An endoscope gas-supply system according to a sixth aspect of the present invention is the endoscope gas-supply system according to any one of the first to fifth aspects of the present invention, further including a gas-supply device which supplies at least an amount of the gas equal to a volume of the gas-supply duct between the buffer tank and the lumen, before the pressure measurement device performs the pressure measurement.

According to the sixth aspect, the pressure measurement device can perform the pressure measurement stably without being affected by intraluminal contents flowing back through the gas-supply duct.

An endoscope gas-supply system according to a seventh aspect of the present invention is the endoscope gas-supply system according to any one of the first to sixth aspects of the present invention, further including a duct resistance varying device which can vary a ratio of a first duct resistance being a resistance of the gas-supply duct on a gas supply source side with respect to the buffer tank, to a second duct resistance being a resistance of the gas-supply duct on a lumen side with respect to the buffer tank.

An endoscope gas-supply system according to an eighth aspect of the present invention is the endoscope gas-supply system according to the seventh aspect of the present invention, further including a duct resistance control device which determines whether or not the intraluminal pressure of the lumen is more than a predetermined threshold value, and which controls the duct resistance varying device so as to make the first duct resistance higher than the second duct resistance when the intraluminal pressure of the lumen is more than the predetermined threshold value.

An endoscope gas-supply system according to a ninth aspect of the present invention is the endoscope gas-supply system according to the eighth aspect of the present invention, wherein the duct resistance control device controls the duct resistance varying device so as to make the first duct resistance lower than the second duct resistance when the intraluminal pressure of the lumen is equal to or less than the predetermined threshold value.

According to the seventh to ninth aspects of the present invention, it becomes possible to stably supply the gas regulated to a predetermined pressure into the lumen, thereby reducing the operation burden on the operator.

Further, in order to achieve the above object, an endoscope gas-supply system according to a tenth aspect of the present invention is an endoscope gas-supply system which supplies gas from a gas supply source into a lumen of a living body, the endoscope gas-supply system including: a gas-supply duct for supplying gas from the gas supply source into the lumen of the living body; a pressure regulation device which regulates a pressure of the gas supplied from the gas supply source; a buffer tank which is provided in the gas-supply duct on a lumen side with respect to the pressure regulation device and which temporarily stores the gas flowing through the gas-supply duct; a pressure measurement device which measures an internal pressure of the buffer tank; a setting device which sets a set value of the intraluminal pressure of the lumen; and a pressure control device which calculates the intraluminal pressure of the lumen from a result of the measurement by the pressure measurement device, and which controls the pressure of the gas regulated by the pressure regulation device such that the intraluminal pressure of the lumen becomes the set value set by the setting device, wherein a first duct resistance of the gas-supply duct on a gas supply source side with respect to the buffer tank is larger than a second duct resistance of the gas-supply duct on a lumen side with respect to the buffer tank.

According to the tenth aspect, the intraluminal pressure fluctuation due to volume variation of the lumen can be effectively absorbed in the buffer tank, and the pressure measurement device can measure the intraluminal pressure of the lumen through the gas-supply duct stably and accurately. In particular, since the first duct resistance of the gas-supply duct between the buffer tank and the gas supply source is made larger than the second duct resistance of the gas-supply duct between the buffer tank and the lumen in the case where the intraluminal pressure of the lumen is measured indirectly by measuring the internal pressure of the buffer tank, it become possible to control the intraluminal pressure of the lumen reliably while reducing a negative effect from the gas supply. This makes it possible to stably supply the gas regulated to a predetermined pressure into the lumen, thereby reducing the operation burden on the operator.

An endoscope gas-supply system according to an eleventh aspect of the present invention is the endoscope gas-supply system according to the tenth aspect of the present invention, further including a duct resistance varying device which can vary a ratio of the first duct resistance to the second duct resistance; and a duct resistance control device which determines whether or not the intraluminal pressure of the lumen is more than a predetermined threshold value, and which controls the duct resistance varying device so as to make the first duct resistance larger than the second duct resistance when the intraluminal pressure of the lumen is more than the predetermined threshold value.

According to the eleventh aspect, since control is made such that the first duct resistance becomes larger than the second duct resistance, depending on the intraluminal pressure of the lumen, it becomes possible to perform the intraluminal pressure control more stably and reliably, thereby significantly reducing the operation burden on the operator.

An endoscope gas-supply system according to a twelfth aspect of the present invention is the endoscope gas-supply system according to the eleventh aspect of the present invention, wherein the duct resistance control device controls the duct resistance varying device so as to make the first duct resistance smaller than the second duct resistance when the intraluminal pressure of the lumen is equal to or less than the predetermined threshold value.

According to the twelfth aspect, since the first duct resistance is made smaller than the second duct resistance when the intraluminal pressure of the lumen is equal to or less than the predetermined threshold value (namely, an initial stage of starting gas supply into the lumen), it become possible to improve the rate of gas supply into the lumen.

An endoscope gas-supply system according to thirteenth aspect of the present invention is the endoscope gas-supply system according to the eleventh or twelfth aspect of the present invention, wherein the duct resistance varying device includes: a plurality of ducts with different duct resistances from each other, the plurality of ducts being arranged in parallel with each other and connected to the gas-supply duct; and a selector valve which can selectively switch among the plurality of ducts so as to make one of the plurality of ducts communicate with the gas-supply duct, and the duct resistance varying device controls the selector valve to cause a desired one of the plurality of ducts to communicate with the gas-supply duct. According to the thirteenth aspect, the duct resistance of the gas-supply duct can be easily varied by selecting one from the plurality of ducts.

An endoscope gas-supply system according to fourteenth aspect of the present invention is the endoscope gas-supply system according to the eleventh or twelfth aspect of the present invention, wherein: the duct resistance varying device includes a control valve which can vary the cross-sectional area of the gas-supply duct; and the duct resistance control device controls the control valve such that the duct resistance of the gas-supply duct becomes a desired value.

According to the fourteenth aspect, since the duct resistance of the gas-supply duct can be varied by varying the cross-sectional area of the control valve, space saving can be achieved as compared with the case where switching among the duct resistances is performed through the use of the plurality of ducts.

An endoscope gas-supply system according to fifteenth aspect of the present invention is the endoscope gas-supply system according to any one of the eleventh to fourteenth aspects of the present invention, wherein the duct resistance varying device is provided in the gas-supply duct on a gas supply source side with respect to the buffer tank.

An endoscope gas-supply system according to sixteenth aspect of the present invention is the endoscope gas-supply system according to any one of the eleventh to fourteenth aspects of the present invention, wherein the duct resistance varying device is provided in the gas-supply duct on a lumen side with respect to the buffer tank.

Like the fifteenth and sixteenth aspects, the duct resistance varying device may be disposed in the gas-supply duct between the buffer tank and the gas supply source, or may be disposed in the gas-supply duct between the buffer tank and the lumen. In each case, it is possible to vary the ratio of the first duct resistance to the second duct resistance, thereby achieving stable pressure control.

An endoscope gas-supply system according to a seventeenth aspect of the present invention is the endoscope gas-supply system according to any one of the tenth to sixteenth aspects of the present invention, wherein the pressure regulation device is provided in the gas-supply duct.

According to the seventeenth aspect, it becomes possible to regulate the pressure of the gas supplied from the gas supply source through the gas-supply duct.

An endoscope gas-supply system according to an eighteenth aspect of the present invention is the endoscope gas-supply system according to any one of the tenth to seventeenth aspects of the present invention, wherein the buffer tank is larger in volume than the lumen.

According to the eighteenth aspect, becomes possible to improve the effect from the buffer tank of reducing the intraluminal pressure fluctuation due to volume variation of the lumen.

According to the present invention, since the intraluminal pressure fluctuation due to volume variation of the lumen can be absorbed effectively in the buffer tank, the pressure measurement device can measure the intraluminal pressure of the lumen stably and reliably through the gas-supply duct. This makes it possible to stably supply the gas regulated to a predetermined pressure into the lumen, thereby reducing the operation burden on the operator.

### BRIEF DESCRIPTION OF THE DRAWINGS

FIG. 1 is a schematic view showing the configuration of an endoscope gas-supply system according to a first embodiment;

FIG. 2 is a perspective view showing a distal end portion of an insertion unit of the endoscope shown in FIG. 1;

FIG. 3 is a side sectional view showing the configuration of an insertion aid shown in FIG. 1;

FIG. 4 is a configuration diagram showing the internal configuration of an gas-supply apparatus shown in FIG. 1;

FIG. 5 is a schematic view showing a first configuration example of a variable volume buffer tank;

FIG. 6 is a schematic view showing a second configuration example of the variable volume buffer tank;

FIG. 7 is a schematic view showing a third configuration example of the variable volume buffer tank;

FIG. 8 is a schematic view showing the configuration of an endoscope gas-supply system according to a second embodiment;

FIG. 9 is a perspective view showing a distal end portion of an insertion unit of the endoscope shown in FIG. 8;

FIG. 10 is a schematic view showing the configuration of an endoscope gas-supply system according to a third embodiment;

FIGS. 11A and 11B are conceptual diagrams showing a difference in effectiveness when duct resistance is variable; and

FIG. 12 is a schematic view showing another configuration of the endoscope gas-supply system according to the third embodiment.

### DETAILED DESCRIPTION OF THE PREFERRED EMBODIMENTS

Hereinafter, preferred embodiments of the present invention will be described with reference to the accompanying drawings.

### First Embodiment

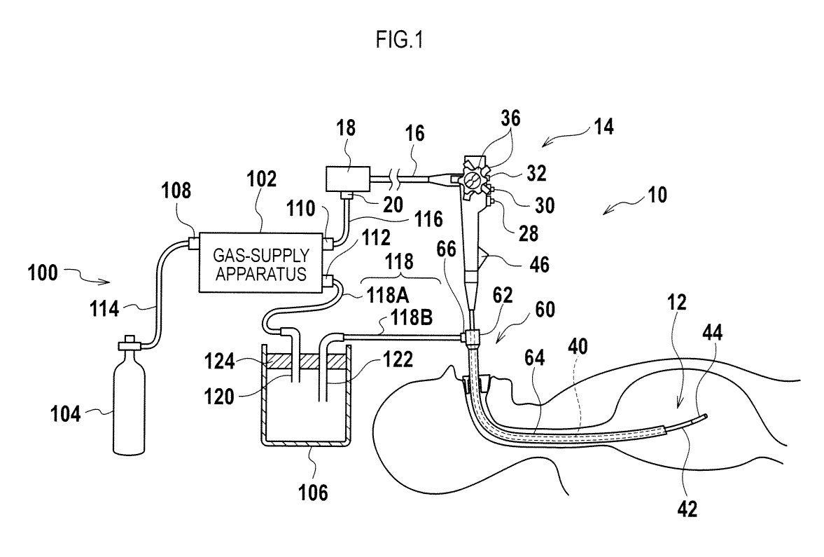

FIG. 1 is a schematic view showing the configuration of an endoscope gas-supply system according to a first embodiment.

As shown in FIG. 1, in the first embodiment, an insertion unit 12 of an endoscope 10, which has been passed through an insertion aid (overtube) 60, is inserted into a lumen orally or per anum to inject a predetermined gas (for example, carbon dioxide gas) supplied from an endoscope gas-supply system 100 described later into the lumen. Here, as an example, the insertion unit 12 and the insertion aid 60 which have been inserted from a patient's mouth into his/her stomach through his/her esophagus are shown, but, not only into the stomach, for example, the insertion unit 12 and the insertion aid 60 may be inserted from a patient's mouth or anus into a lumen, such as the esophagus, the small intestine (the duodenum, the jejunum, the ileum) or the large intestine (the cecum, the colon, the rectum).

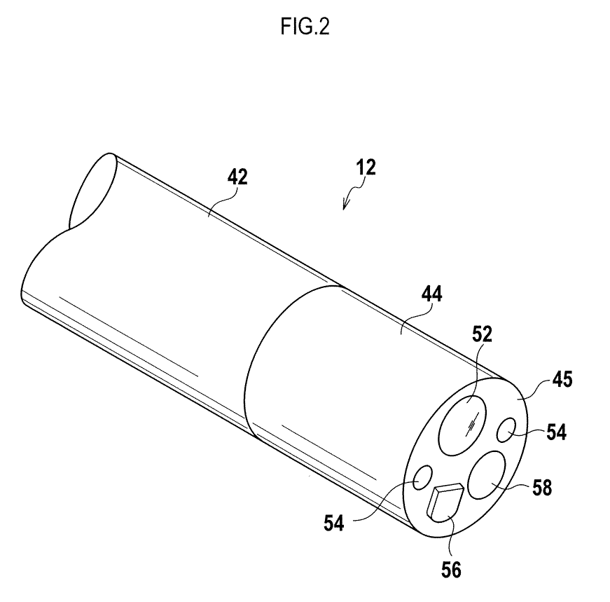

The endoscope 10 is provided with a hand operation unit 14, and the insertion unit 12 which is provided so as to be continuous with the hand operation unit 14 and which is inserted into a human lumen. The hand operation unit 14 is connected with a universal cable 16, and the universal cable 16 is provided with an LG connector 18 at its tip end. This LG connector 18 is attachably and detachably coupled with a light source device (not shown) by which illumination light is transmitted to an illumination optical system 54 (see FIG. 2). Further, the LG connector 18 is connected with an electrical connector, and this electrical connector is attachably and detachably coupled with a processor that performs image signal processing or the like.

The hand operation unit 14 is also provided with a gas-supply/water-supply button 28, a suction button 30, and a shutter button 32, and further provided with a pair of angle knobs 36 and 36.

The insertion unit 12 includes a flexible portion 40, a bending portion 42, and a distal end portion 44 in this order from the hand operation unit 14, and the bending portion 42 is remotely bent by turning the angle knobs 36 and 36 of the hand operation unit 14. This makes it possible to point the distal end portion 44 in a desired direction.

As shown in FIG. 2, a distal end face 45 of the distal end portion 44 is provided with an observation optical system 52, an illumination optical system 54 and 54, a gas-supply/water-supply nozzle 56, and a forceps opening 58. A CCD (not shown) is disposed behind the observation optical system 52, and a signal cable (not shown) is connected to a substrate which supports this CCD. The signal cable is extended up to the electrical connector through the insertion unit 12, the hand operation unit 14, and the universal cable 16 shown in FIG. 1, and connected to the processor. Therefore, an observation image captured by the observation optical system 52 is imaged on a light-receiving surface of the CCD and converted into an electrical signal, and then this electrical signal is outputted to the processor through the signal cable, and converted into a video signal. In this manner, the observation imaged is displayed on a monitor connected to the processor.

A light exit end of a light guide (not shown) is disposed behind the illumination optical systems 54 and 54 in FIG. 2. This light guide is passed through the insertion unit 12, the hand operation unit 14, and the universal cable 16 in FIG. 1, a light entrance end of the light guide is disposed in the LG connector 18. Therefore, by coupling the LG connector 18 with the light source device, the illumination light emitted from the light source device is transmitted to the illumination optical systems 54 and 54 through the light guide, and emitted forward from the illumination optical systems 54 and 54.

The gas-supply and water-supply nozzle 56 in FIG. 2 communicates with a valve (not shown) operated by the gas-supply and water-supply button 28 in FIG. 1, and further this valve communicates with a gas-supply and water-supply connector (not shown) provided in the LG connector 18. The gas-supply and water-supply connector is connected with a gas-supply and water-supply device not shown, and supplied with air or water. Therefore, by operating the gas-supply and water-supply button 28, air or water can be sprayed from the gas-supply and water-supply nozzle 56 toward the observation optical system 52.

As shown in FIG. 1, the LG connector 18 of the first embodiment is provided with a supply port 20 for carbon dioxide gas supplied from a gas-supply apparatus 102 described later. The gas-supply and water-supply device which, though not shown, is connected to the LG connector 18 supplies air or water selectively to a gas-supply and water-supply duct (not shown) of the endoscope 10 through the use of carbon dioxide gas supplied from the gas-supply apparatus 102 through the supply port 20, and sprays air or water from the gas-supply and water-supply nozzle 56. Incidentally, the configuration of the gas-supply and water-supply device is publicly known (for example, see Japanese Patent Application Laid-Open No. 2009-153641 or Japanese Patent Application Laid-Open No. 2009-022444), and therefore is not described in detail here.

The forceps opening 58 in FIG. 2 communicates with a forceps insertion unit 46 in FIG. 1 through a forceps channel (not shown). Therefore, by inserting a treatment instrument, such as forceps, from the forceps insertion unit 46, this treatment instrument can be guided out of the forceps opening 58. Further, the forceps opening 58 communicates with a valve (not shown) operated by the suction button 30, and further this valve is connected to a suction connector (not shown) of the LG connector 18. Therefore, by connecting a suction device, not shown, to the suction connector and operating the valve by the suction button 30, a lesion or the like can be sucked from the forceps opening 58.

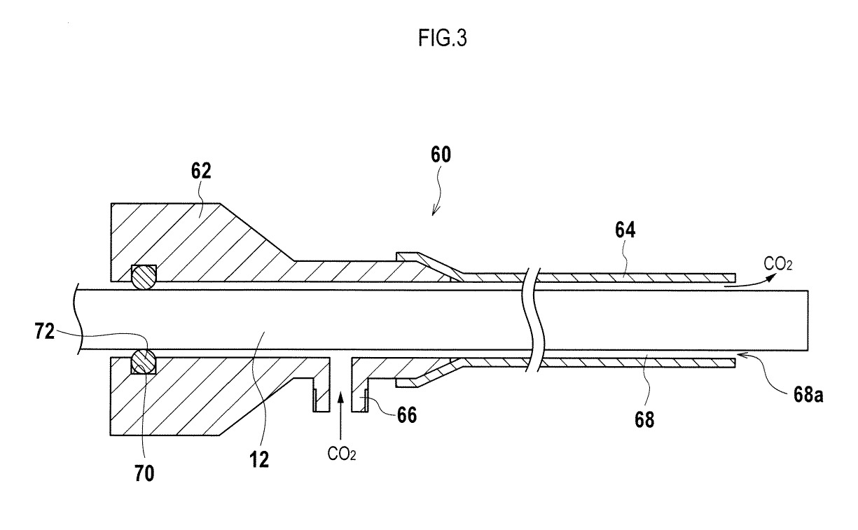

On the other hand, as shown in FIG. 3, the insertion aid 60 shown in FIG. 1 is provided with a holding portion 62 and a tube main body 64. The tube main body 64 is formed into a tubular shape, and has an inner diameter larger than the outer diameter of the insertion unit 12 such that the insertion unit 12 of the endoscope 10 can be passed through the tubular main body 64. Further, the tube main body 64 is a flexible urethane-resin molded product, and an outer periphery of the tube main body 64 is covered with a lubrication coating, and an inner periphery thereof is also covered with a lubrication coating. The tube main body 64 is fitted in a water-tight manner on the hard holding portion 62 shown in FIG. 1, and the holding portion 62 is attachably and detachably coupled with the tube main body 64. The insertion unit 12 is inserted in the tube main body 64 through a proximal end opening portion of the holding portion 62.

On a proximal end side of the insertion aid 60, a supply port 66 for injecting carbon dioxide gas is provided. This supply port 66 is opened in an inner periphery of the insertion aid 60, and communicates with an insertion passage 68 formed inside the insertion aid 60. Further, on a proximal end side with respect to the supply port 66 of the insertion aid 60 (in other words, at a position between the proximal end and the supply port 66) an annular groove portion 70 is formed in a circumferential direction in an inner periphery of the insertion passage 68, and an O-ring 72 is provided in the groove portion 70 as an air-tight device which prevents leakage of gas supplied from the supply port 66. This makes it possible to supply the gas supplied from the gas-supply apparatus 102 described later to the insertion passage 68 of the insertion aid 60 through the supply port 66, and introduce the gas into the lumen through a distal end opening portion 68a of the insertion aid 60 without allowing any gas to leak from the proximal end side, thereby inflating the lumen.

Thus, in the first embodiment, the insertion passage 68 formed inside the insertion aid 60 (specifically, a space formed between an inner wall surface of the insertion passage 68 and the insertion unit 12) functions as a part of a gas-supply duct for automatic gas-supply of the gas supplied from the gas-supply apparatus 102 into the lumen.

Next, the configuration of the endoscope gas-supply system 100, which is a characteristic part of the present invention, will be described.

As shown in FIG. 1, the endoscope gas-supply system 100 is mainly composed of the gas-supply apparatus 102 which is a gas supply device, a carbon dioxide gas cylinder 104 reserving liquefied carbon dioxide gas, and a buffer tank 106.

The gas-supply apparatus 102 is a device which supplies carbon dioxide gas into the lumen through a gas-supply duct provided in the insertion unit 12 of the endoscope 10 or the insertion aid 60 while reducing the pressure of carbon dioxide gas supplied from the carbon dioxide gas cylinder 104 to a predetermined pressure. The gas-supply apparatus 102 is provided with a high-pressure connector 108 for inputting carbon dioxide gas supplied from the carbon dioxide gas cylinder 104, and first and second gas-supply connectors 110 and 112 for outputting carbon dioxide gas so regulated by the gas-supply apparatus 102 as to have a predetermined pressure.

The high-pressure connector 108 is coupled with one end of a tube for high-pressure gas 114, and the other end of the tube for high-pressure gas 114 is coupled with the carbon dioxide gas cylinder 104. The liquefied carbon dioxide gas stored in the carbon dioxide gas cylinder 104 is vaporized and introduced into the gas-supply apparatus 102 through the tube for high-pressure gas 114 and the high-pressure connector 108. Though an internal configuration of the gas-supply apparatus 102 will be described later, the carbon dioxide gas inputted from the high-pressure connector 108 is subjected to various treatments, and then outputted from the first and second gas-supply connectors 110 and 112.

The first gas-supply connector 110 is coupled with one end of a first gas-supply tube 116, and the other end of the first gas-supply tube 116 is coupled with the supply port 20 of the LG connector 18. The carbon dioxide gas which has a pressure reduced to a predetermined pressure by the gas-supply apparatus 102 is introduced into the LG connector 18 through the first gas-supply connector 110, the first gas-supply tube 116, and the supply port 20. Then, the carbon dioxide gas is supplied to the gas-supply and water-supply device which, though not shown, is connected to the LG connector 18, and, according to the operation of the gas-supply and water-supply button 28, air or water is sprayed from the gas-supply and water-supply nozzle 56 of the insertion unit 12 of the endoscope 10.

The second gas-supply connector 112 is coupled with one end of the second gas-supply tube 118, and the other end of the second gas-supply tube 118 is coupled with the supply port 66 provided at the proximal end of the insertion aid 60. The carbon dioxide gas which has a pressure reduced to a predetermined pressure by the gas-supply apparatus 102 is supplied to the insertion passage 68 in the insertion aid 60 through the second gas-supply connector 112, the second gas-supply tube 118, and the supply port 66, and the carbon dioxide gas is introduced into the lumen from the distal end opening portion 68a of the insertion aid 60 through the passage 68.

In the first embodiment, the second gas-supply tube 118 is mainly composed of an upstream (the gas-supply apparatus 102 side) gas-supply tube 118A and a downstream (the luminal side) gas-supply tube 118B, and the buffer tank 106 is disposed therebetween. The buffer tank 106 is provided with a gas feed pipe 120 serving as a gas feed duct, a gas discharge pipe 122 serving as an air discharge duct, and a lid member 124 closing a top opening of the buffer tank 106. The volume of the buffer tank 106 (the volume of a space closed by the lid member 124 in the buffer tank 106) is configured to be larger than that of the lumen so that the buffer tank 106 serves to absorb the intraluminal pressure fluctuation due to volume variation of the lumen.

The second gas-supply connector 112 is coupled with one end of the upstream gas-supply tube 118A, and the other end of the upstream gas-supply tube 118A is coupled with an upper end of the gas feed pipe 120 of the buffer tank 106. The gas feed pipe 120 is disposed so as to penetrate the lid member 124 of the buffer tank 106, and a lower end of the gas feed pipe 120 is disposed, in the buffer tank 106, apart from a bottom face of the buffer tank 106. This causes gas (that is, carbon dioxide gas) supplied from the gas-supply apparatus 102 through the upstream gas-supply tube 118A to be temporarily stored (reserved) in the buffer tank 106.

The gas discharge pipe 122, similarly to the gas feed pipe 120, is disposed so as to penetrate the lid member 124, and a lower end of the gas discharge pipe 122 is disposed, in the buffer tank 106, apart from the bottom face of the buffer tank 106. An upper end of the gas discharge pipe 122 is coupled with one end of the downstream gas-supply tube 118B, and the other end of the downstream gas-supply tube 118B is coupled with the supply port 66 of the insertion aid 60. This causes the gas stored temporarily in the buffer tank 106 to be fed to the downstream gas-supply tube 118B through the gas discharge pipe 122, and then introduced into the lumen through the supply port 66 and the insertion passage 68 in the insertion aid 60.

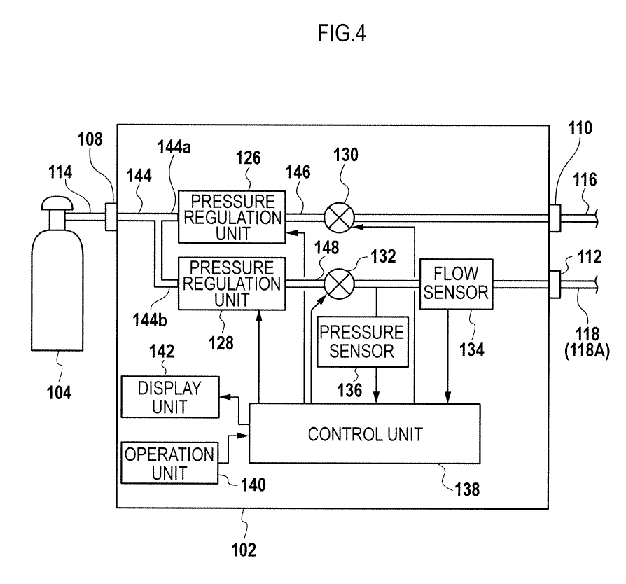

Here, the internal configuration of the gas-supply apparatus 102 is shown in FIG. 4. As shown in FIG. 4, the gas-supply apparatus 102 is provided with first and second pressure regulation units 126 and 128, first and second solenoid valves 130 and 132, a flow sensor 134, a pressure sensor 136, a control unit 138, an operation unit 140, and a display unit 142.

The first and second pressure regulation units 126 and 128 are connected to the high-pressure connector 108 through a bifurcated internal duct 144. Each of the first and second pressure regulation units 126 and 128 regulates the pressure of carbon dioxide gas inputted from the high-pressure connector 108 through the internal duct 144, and sends out the carbon dioxide gas after pressure regulation into internal ducts 146 and 148, respectively, on their output sides.

In the first embodiment, each of the first and second pressure regulation units 126 and 128 is composed of a pressure control valve that can perform pressure control electrically, and controls the pressure of carbon dioxide gas based on a control signal from the control unit 138.

The internal duct 146 is a duct for guiding the carbon dioxide gas fed from the first pressure regulation unit 126 to the first gas-supply connector 110. In the course of the internal duct 146, the first solenoid valve 130 for selectively performing supply/interruption of supply of the carbon dioxide gas is provided. The first solenoid valve 130 is an on-off valve that can electrically open and close the duct (that is, the internal duct 146), and is opened or closed based on a control signal from the control unit 138.

The internal duct 148 is a duct for guiding the carbon dioxide gas fed from the second pressure regulation unit 128 to the second gas-supply connector 112. In the course of the internal duct 148, the second solenoid valve 132 for selectively performing supply/interruption of supply of the carbon dioxide gas is provided. The second solenoid valve 132, similarly to the first solenoid valve 130, is an on-off valve that can electrically open and close the duct (that is, the internal duct 148), and is opened or closed based on a control signal from the control unit 138.

The flow sensor 134 is a sensor that detects the flow rate of carbon dioxide gas flowing in the internal duct 148, and the detection result is outputted to the control unit 138.

The pressure sensor 136 is a sensor that detects pressure in the internal duct 148, and the detection result is outputted to the control unit 138.

It should be noted that, though the pressure sensor 136 is set for the purpose of measuring the intraluminal pressure of the lumen, it is impossible to set a universal (general-purpose) pressure sensor in a lumen to measure the intraluminal pressure of the lumen directly. Therefore, the intraluminal pressure of the lumen is measured indirectly by the pressure sensor 136 through a gas-supply duct (namely, the internal duct 148, the second gas-supply tube 118, and the passage 68) for automatic gas supply from the gas-supply apparatus 102 into the lumen. If a pressure measured by the pressure sensor 136 (that is, the pressure in the internal duct 148) is represented by P1, an intraluminal pressure of the lumen by P2, and a pressure loss in the gas-supply duct therebetween by ΔP, then the intraluminal pressure of the lumen P2 is obtained as P1−ΔP. At this time, it is preferred that the pressure loss ΔP for each combination of the endoscope 10, the insertion aid 60, the gas-supply tube, and the like used in practice is preliminarily obtained, and these data are tabularized, and stored as pressure loss data in a memory device (not shown). This makes it possible to easily obtain the intraluminal pressure of the lumen P2 from the pressure of the internal duct 148 P1 measured by the pressure sensor 136.

It should be noted that, though the pressure of the internal duct 148 in the gas-supply apparatus 102 is indirectly measured by the pressure sensor 136 in the first embodiment, a location where the intraluminal pressure of the lumen is indirectly measured is not particularly limited as long as it is in a duct communicating with the lumen, and, for example, a pressure sensor that detects pressure in the buffer tank 106 may be provided so that the pressure of carbon dioxide gas supplied from the gas-supply apparatus 102 is controlled according to the detection result of the pressure sensor.

The control unit 138 performs various controls based on an operation signal from the operation unit 140 described later and detection signals from the flow sensor 134 and the pressure sensor 136. Particularly in the first embodiment, the control unit 138 controls the first and second solenoid valves 130 and 132 based on the operation signal from the operation unit 140, and controls the pressure of carbon dioxide gas so regulated by the second pressure control unit 128 so as to become a set value, based on the detection signals from the flow sensor 134 and the pressure sensor 136.

The operation unit 140 is provided with operation buttons for performing various settings of the gas-supply apparatus 102. Particularly in the first embodiment, the operation unit 140 is provided with an operation button (carbon dioxide gas feeding/feeding interruption button) for instructing feeding/feeding interruption of the carbon dioxide gas from the first and second gas-supply connectors 110 and 112, and an operation button (pressure setting button) for setting an intraluminal set pressure. When any of the operation buttons is operated by the operator or the like, an operation signal corresponding to an operation content of the operation button is outputted to the control unit 138.

The display unit 142 displays setting and operation conditions of the gas-supply apparatus 102, and displays the condition of feeding (feeding/feeding interruption) of the carbon dioxide gas from the first and second gas-supply connectors 110 and 112, the intraluminal set pressure, a current intraluminal pressure of the lumen, or the like, based on control signals from the control unit 138.

Next, the operations of the endoscope gas-supply system 100 of the first embodiment will be described.

First, when the gas-supply apparatus 102 is powered on, the control unit 138 performs operation check or initial setup of each unit or portion. Then, when an operation button for instructing start of manual gas supply and/or automatic gas supply is pressed, the first or second solenoid valve 130 or 132 is opened according to the operation content of the operation button. Specifically, when the manual gas supply is selected, the first solenoid valve 130 is opened so that the gas can be fed through the first gas-supply connector 110. When the automatic gas supply is selected, the second solenoid valve 132 is opened so that the gas can be fed through the second gas-supply connector 112. When both the manual gas supply and the automatic gas supply are selected, the first and second solenoid valves 130 and 132 are opened so that the gas can be fed through the first and second gas supply connectors 110 and 112.

The carbon dioxide gas supplied from the carbon dioxide gas cylinder 104 to the gas-supply apparatus 102 through the tube for high-pressure gas 114 and the high-pressure connector 108 is bifurcated by the internal duct 144: one bifurcated flow of the carbon dioxide gas is inputted into the first pressure regulation unit 126 through a bifurcated duct 144a; and the other bifurcated flow thereof is inputted into the second pressure regulation unit 128 through a bifurcated duct 144b.

In the first pressure regulation unit 126, the pressure of the carbon dioxide gas inputted through the bifurcated duct 144a of the internal duct 144 is reduced to a predetermined pressure. At this time, the pressure of the carbon dioxide gas after the pressure reduction may be regulated according to the control signal from the control unit 138. The carbon dioxide gas which has a pressure reduced to a predetermined pressure is outputted to the internal duct 146 so that feeding of the carbon dioxide gas through the first gas-supply connector 110 is performed. It should be noted that, when the first solenoid valve 130 is closed, the carbon dioxide gas is not fed through the first gas-supply connector 110.

The carbon dioxide gas fed through the first gas-supply connector 110 is introduced to the LG connector 18 through the first gas-supply tube 116 and the supply port 20. Then, the carbon dioxide gas is supplied to the gas-supply and water-supply device, not shown, connected to the LG connector 18, and, according to the operation of gas-supply and water-supply button 28, the carbon dioxide gas is introduced into the lumen through the gas-supply and water-supply nozzle 56 of the insertion unit 12 of the endoscope 10.

On the other hand, in the second pressure regulation unit 128, the pressure of the carbon dioxide gas inputted through the bifurcated duct 144b of the internal duct 144 is reduced to a predetermined pressure, and the carbon dioxide gas after the pressure reduction is outputted to the internal duct 148. At this time, the pressure inside the internal duct 148 is detected by the pressure sensor 136, and the detection result is outputted to the control unit 138. The control unit 138 calculates the intraluminal pressure of the lumen from the detection result provided by the pressure sensor 136, and sends a control signal to the second pressure regulation unit 128 to cause the intraluminal pressure of the lumen to become a set pressure set by the operation unit 140. The second pressure regulation unit 128 regulates the opening degree of the pressure control valve based on the control signal provided by the control unit 138. While being subjected to feedback control according to the detection result of the pressure sensor 136 so as to become the predetermined pressure, the carbon dioxide gas outputted from the second pressure regulation unit 128 to the internal duct 148 in this manner is fed through the second gas-supply connector 112. It should be noted that, when the second solenoid valve 132 is closed, the carbon dioxide gas is not fed through the second gas-supply connector 112.

The carbon dioxide gas fed through the second gas-supply connector 112 is introduced to the insertion passage 68 in the insertion aid 60 through the second gas-supply tube and the supply port 66. Then, the carbon dioxide gas is introduced into the lumen through the distal end opening portion 68a of the insertion aid 60.

Here, in the first embodiment, as described above, the buffer tank 106 which is larger in volume than the lumen is interposed in the second gas-supply tube 118, and the carbon dioxide gas fed through the second gas-supply connector 112 is temporarily stored in the buffer tank 106 through the upstream gas-supply tube 118A and the gas feed pipe 120, and introduced into the lumen through the gas discharge pipe 122, the downstream gas-supply tube 118B, the supply port 66, and the insertion passage 68 of the insertion aid 60.

Thus, since the buffer tank 106 is provided in the gas-supply duct (namely, the second gas-supply tube 118) for automatic gas supply of the carbon dioxide gas from the gas-supply apparatus 102 into the lumen, the intraluminal pressure fluctuation due to volume variation of the lumen can be reduced by the buffer tank 106, so that stable pressure measurement by the pressure sensor 136 provided in the gas-supply apparatus 102 becomes possible. This makes it possible to perform automatic gas supply from the gas-supply apparatus 102 into the lumen more stably.

Further, the buffer tank 106 of the first embodiment has a trapping structure that can trap luminal contents (for example, filth, residues, or the like) (that is, a structure that can perform gas-liquid separation) so that the buffer tank 106 can prevent the content from entering the gas-supply apparatus 102 even if the contents flow back through the gas-supply duct. Further, since the volume of the buffer tank 106 (the volume of a space closed by the lid member 124 in the buffer tank 106) is configured to be larger than the luminal volume, it is possible to absorb the intraluminal pressure fluctuation due to volume variation of the lumen, thereby performing gas supply based on precise pressure measurement.

Further, by performing the pressure measurement after supplying the carbon dioxide gas of a volume equal to the volume of the gas-supply duct located downstream (on the side of the lumen) of the buffer tank 106, the pressure measurement can avoid an effect from the backflow contents.

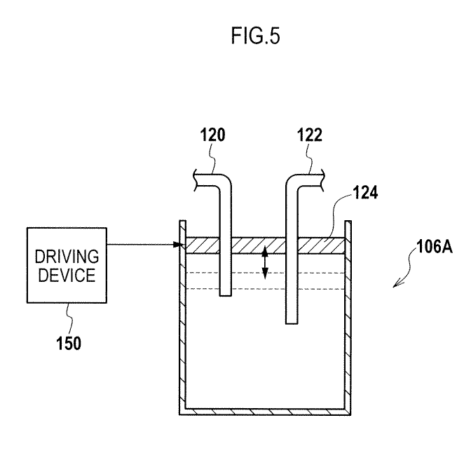

In the first embodiment, it is preferred that the buffer tank 106 is a variable volume buffer tank that increases and decreases in volume according to the volume of the lumen. Here, FIGS. 5 to 7 show configuration examples of the variable volume buffer tank. It should be noted that, in FIGS. 5 to 7, members in common with or similar to those in FIG. 1 are denoted by the same reference numerals in order to omit to describe the members.

In a variable volume buffer tank 106A shown in FIG. 5, a lid 124 is configured to be capable of being moved vertically by a driving device 150. The volume of the variable volume buffer tank 106A increases as the lid 124 moves upward, while the volume thereof decreases as the lid 124 moves downward.

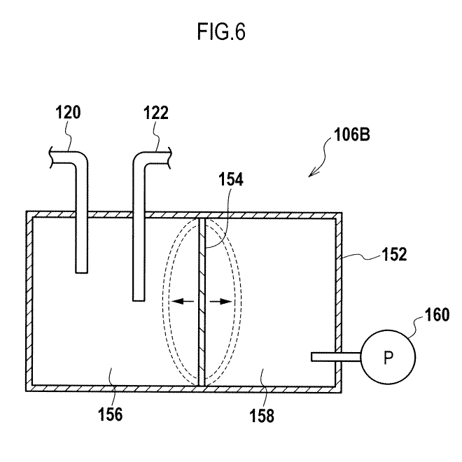

In a variable volume buffer tank 106B shown in FIG. 6, an internal space of an airtight container 152 is partitioned into two spaces 156 and 158 by an elastically-deformable elastic film 154. Of these spaces 156 and 158, the first space 156 communicates with the lower ends of the pipes 120 and 122 so as to function as a buffer tank that temporarily stores the gas supplied from the gas-supply apparatus 102 (see FIG. 1). On the other hand, the second space 158 is connected with a pump 160, and the pump 160 is driven to vary the pressure in the second space 158, thereby deforming the elastic film 154. This causes the volume of the first space 156 to increase and decrease. It should be noted that the second space 158 is filled with, but not limited to, gas, and may be filled with liquid.

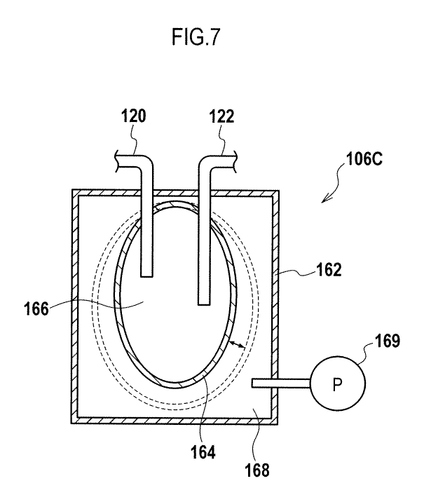

In a variable volume buffer tank 106C shown in FIG. 7, an elastically-deformable bag-like member 164 is provided in an airtight container 162. A space 166 in the hag-like member 164 communicates with the lower ends of the pipes 120 and 122, and functions as a buffer tank that temporarily stores the gas supplied from the gas-supply apparatus 102 (see FIG. 1). On the other hand, a pump 169 is connected to a space 168 inside the airtight container 162 and outside the hag-like member 164, and the pump 169 is driven to vary the pressure in the space 168, thereby increasing and decreasing the volume of the space 166 in the bag-like member 164. It should be noted that the space 168 is filled with, but not limited to, gas, and may be filled with liquid.

Thus, since any of the variable volume buffer tanks 106A to 106C that increase and decrease in volume according to the volume of the lumen is used, the intraluminal pressure fluctuation due to volume variation of the lumen can effectively be absorbed.

As described above, according to the first embodiment, automatic gas-supply (constant-pressure gas-supply) into the lumen is constantly performed with the intraluminal pressure of the lumen controlled to be a set pressure, while the intraluminal pressure of the lumen is being indirectly measured by the pressure sensor 136 in the gas-supply apparatus 102 through the gas-supply duct for automatic gas supply. At this time, since the gas-supply duct is provided with the buffer tank 106 which is larger in volume than the lumen, the buffer tank 106 can absorb the intraluminal pressure fluctuation due to volume variation of the lumen. Therefore, it becomes possible to perform automatic gas supply into the lumen stably, thereby lightening the operation burden on the operator.

Further, in the first embodiment, the carbon dioxide gas supplied from the gas-supply apparatus 102 can be supplied into the lumen through the gas-supply and water-supply duct (not shown) of the endoscope 10 manually according to operator's operation. This makes it possible to perform fine adjustment (fine regulation) of the intraluminal pressure of the lumen to a desired pressure according to operator's manipulation, thereby keeping the inside of the lumen in an appropriate condition in combination with the automatic gas supply.

It should be noted that, in the first embodiment, the insertion passage 68 formed in the insertion aid 60 is utilized as part of the gas-supply duct for automatic gas supply, but this is not a limitation and, for example, a duct for automatic gas supply may be provided integrally with or separately from the insertion passage 68 of the insertion aid 60.

### Second Embodiment

Next, a second embodiment of the present invention will be described. Hereinafter, a description of members in common with the first embodiment will be omitted, and a feature of the second embodiment will be mainly described.

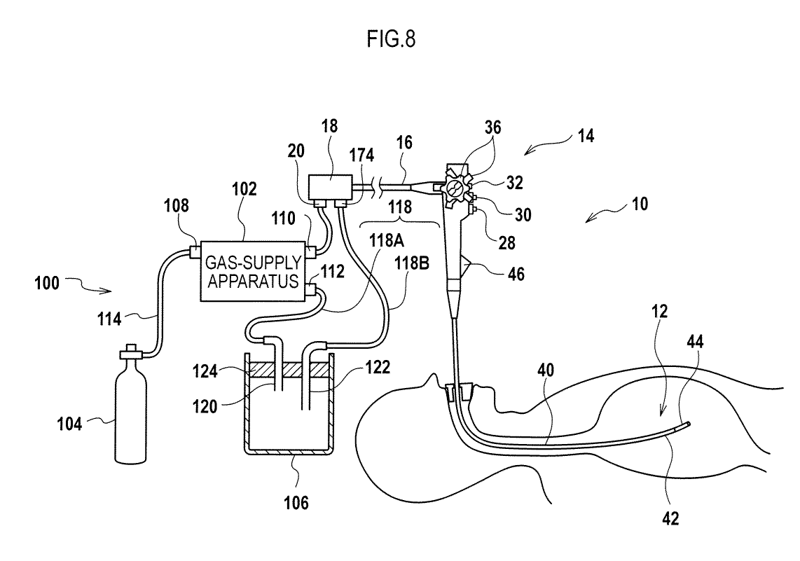

FIG. 8 is a schematic view showing the configuration of an endoscope gas-supply system according to the second embodiment.

In the first embodiment, with the insertion unit 12 of the endoscope 10 which has passed through the insertion aid 60, the insertion aid 60 and the insertion unit 12 are inserted into the lumen, but, in the second embodiment, as shown in FIG. 8, the insertion unit 12 of the endoscope 10 is independently inserted into the lumen.

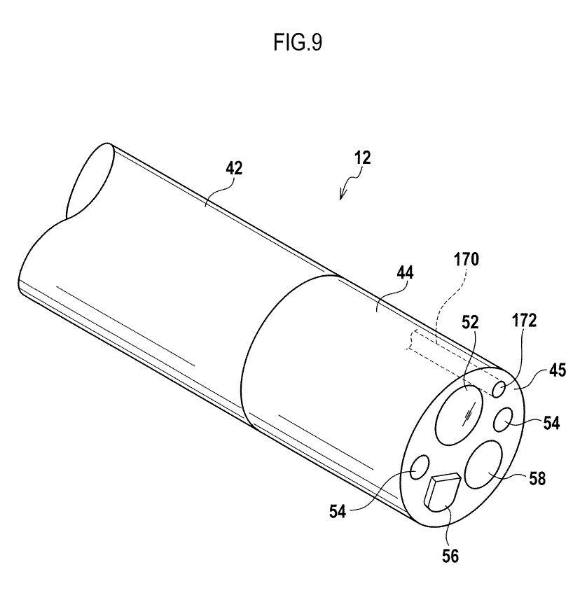

As shown in FIG. 9, the insertion unit 12 of the endoscope 10 is provided with a gas-supply duct (duct for automatic gas supply) 170 for performing automatic gas supply into the lumen while measuring the intraluminal pressure of the lumen indirectly. An opening (opening for automatic gas supply) 172 communicating with the duct for automatic gas supply 170 is formed in a distal end face 45 of the insertion unit 12. The duct for automatic gas supply 170 extends from the insertion unit 12 to the LG connector 18 through the hand operation unit 14 and the universal cable 16, and communicates with a supply port 174 of the LG connector 18. The supply port 174 is connected with the second gas-supply connector 112 of the gas-supply apparatus 102 through the second gas-supply tube 118. The second gas-supply tube 118 is mainly composed of the upstream gas-supply tube 118A and the downstream gas-supply tube 118B, and the buffer tank 106 is disposed therebetween like the first embodiment. The other configurations are the same as those in the first embodiment.

According to the second embodiment, since the insertion unit 12 of the endoscope 10 is provided with the duct for automatic gas supply 170 and the opening for automatic gas-supply 172, it becomes possible to guide gas (that is, carbon dioxide gas) automatically supplied from the gas-supply apparatus 102 into the lumen, without using the insertion aid 60 (see FIG. 1), such as one in the first embodiment.

Further, since the buffer tank 106 is provided in the gas-supply duct for automatic gas supply from the gas-supply apparatus 102 into the lumen, it becomes possible to measure the intraluminal pressure of the lumen by the pressure sensor 136 (FIG. 4) in the gas-supply apparatus 102 without an effect from the intraluminal pressure fluctuation due to volume variation of the lumen, thereby performing automatic gas supply into the lumen stably and reliably.

### Third Embodiment

Next, a third embodiment of the present invention will be described. Hereinafter, a description of a member in common with the first embodiment will be omitted, and a feature of the third embodiment will be mainly described.

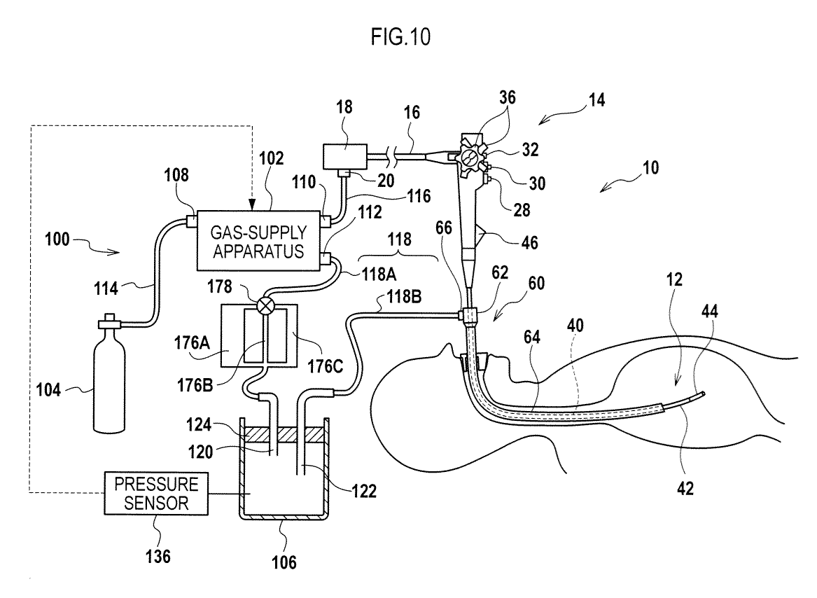

FIG. 10 is a schematic view showing the configuration of an endoscope gas-supply system according to the third embodiment. In FIG. 10, components in common with or similar to FIG. 1 or FIG. 4 are denoted by the same reference numerals.

In the third embodiment, as shown in FIG. 10, the pressure sensor 136 detects the pressure in the buffer tank 106, and the detection result is outputted to the control unit 138 (see FIG. 4) of the gas-supply apparatus 102.

Further, a plurality of (in the this example, three) ducts 176A to 176C having different duct resistances are connected, with arranged in parallel with each other, to the upstream gas-supply tube 118A of the second gas-supply tube 118. A selector valve is provided at an upstream bifurcation point of these ducts 176A to 176C. The selector valve 178 is, for example, composed of an electrically-switchable solenoid valve (four-way valve), and can selectively switch among the ducts 176A to 176C according to the control signal provided by the control unit 138 of the gas-supply apparatus 102 so as to make one of the ducts 176A to 176C communicate with the upper gas-supply tube 118A. It should be noted that FIG. 10 shows the ducts 176A to 176C schematically because of limitations on the drawing, but in practice the shape and size (length and thickness) of each duct is determined so as to have a desired duct resistance value. The other configurations are the same as those in the first embodiment.

According to the third embodiment, automatic gas supply into the lumen is constantly performed with the intraluminal pressure of the lumen controlled to a set pressure, while the pressure in the buffer tank 106 is being measured by the pressure sensor 136. At this time, the pressure control is performed in consideration of a pressure loss between the buffer tank 106 and the inside of the lumen.

Further, the plurality of ducts 176A to 176C which have different duct resistances from each other and which are arranged in parallel with each other are connected to the upstream gas-supply tube 118A constituting a part of the gas-supply duct for automatic gas supply into the lumen, and the ducts 176A to 176C can be selectively switched from one another by the selector valve 178 so that one of the ducts 176A to 176C communicates with the internal duct 148. This makes it possible to vary the ratio of a duct resistance A between the buffer tank 106 and the gas-supply apparatus 102 to a duct resistance B between the buffer tank 106 and the inside of the lumen. Therefore, in a normal state after the intraluminal pressure of the lumen reaches the set pressure, by making the duct resistance A higher than the duct resistance B, the pressure control for automatic gas supply can be performed more stably and reliably while reducing a negative effect of gas supply from the gas-supply apparatus 102.

Further, it is preferred that, in an early stage of pressure application before the intraluminal pressure of the lumen reaches the set pressure, the rate of gas supply is increased by making the duct resistance A lower than the duct resistance B, and after the intraluminal pressure of the lumen reaches the set pressure, stable pressure control is performed with the duct resistance A made higher than the duct resistance B.

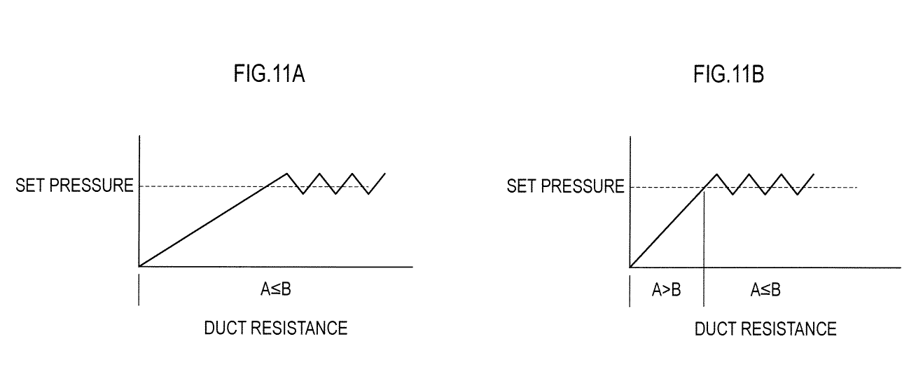

FIGS. 11A and 11B are conceptual diagrams showing a difference in effectiveness when the ratio of the duct resistance A to the duct resistance B is varied, FIG. 11A showing a case where control has been made such that the state where the duct resistance A≦the duct resistance B is satisfied is kept from the early stage of pressure application, and FIG. 11B showing a case where control has been made such that the state where the duct resistance A>the duct resistance B is satisfied in the early stage of pressure application, and then the state is switched to satisfy the duct resistance A≦the duct resistance B when the intraluminal pressure of the lumen reaches the set pressure. As understood from these figures, since the intraluminal pressure of the lumen in FIG. 11B reaches the set pressure faster than that in FIG. 11A, it becomes possible to perform stable pressure control reliably.

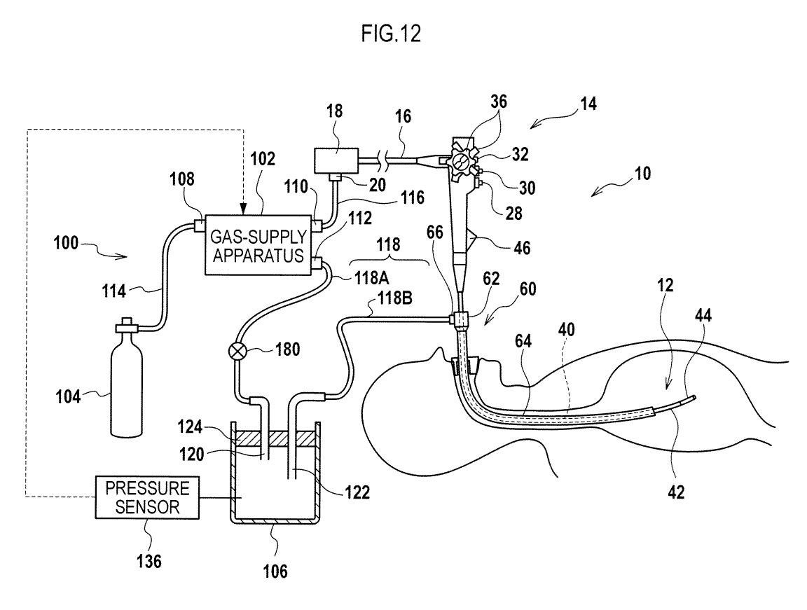

It should be noted that, in the third embodiment, the plurality of ducts 176A to 176C having different duct resistances from each other are provided as a duct resistance varying device, but the duct resistance varying device is not limited to these ducts. For example, as shown in FIG. 12, there is also a preferred embodiment where the upstream gas-supply tube 118A is provided with a flow control valve (variable orifice) 180 so that the duct resistance can be varied by varying the opening degree of the flow control valve 180. According to this preferred embodiment, space-saving is achieved as compared with the third embodiment where the plurality of ducts 176A to 176C are used, and it becomes possible to achieve downsizing of the endoscope gas-supply system 100.

Further, the duct resistance varying device may be provided in the downstream gas-supply tube 118B instead of the upstream gas-supply tube 118A, and the same effect can be obtained.

Hereinabove, the endoscope gas-supply system according to the present invention has been described in detail, but the present invention is not limited to the above examples, and obviously can be modified or varied variously without departing from the scope of the invention.

What is claimed is:

1. An endoscope gas-supply system which supplies gas from a gas supply source into a lumen of a living body, the endoscope gas-supply system comprising:
a gas-supply duct which supplies gas from the gas supply source into the lumen of the living body;
a pressure regulation device which regulates a pressure of the gas supplied from the gas supply source;
a pressure measurement device which measures an intraluminal pressure of the lumen through the gas-supply duct;
a setting device which sets a set value of the intraluminal pressure of the lumen;
a pressure control device which controls the pressure of the gas regulated by the pressure regulation device based on a result of the measurement by the pressure measurement device such that the intraluminal pressure of the lumen becomes the set value set by the setting device; and
a buffer tank which is provided in the gas-supply duct and which temporarily stores the gas flowing through the gas-supply duct.

2. The endoscope gas-supply system according to claim 1 wherein the pressure regulation device is provided in the gas-supply duct.

3. The endoscope gas-supply system according to claim 1, wherein the buffer tank is larger in volume than the lumen.

4. The endoscope gas-supply system according to claim 1, wherein the buffer tank is a variable volume buffer tank which increases and decreases in volume according to the volume of the lumen.

5. The endoscope gas-supply system according to claim 1, wherein the buffer tank has a gas-liquid separation structure which separates gas and liquid flowing through the gas-supply duct from each other.

6. The endoscope gas-supply system according to claim 1, further comprising
a gas-supply device which supplies at least an amount of the gas equal to a volume of the gas-supply duct between the buffer tank and the lumen, before the pressure measurement device performs the pressure measurement.

7. The endoscope gas-supply system according to claim 1, further comprising
a duct resistance varying device which can vary a ratio of a first duct resistance being a resistance of the gas-supply duct on a gas supply source side with respect to the buffer tank, to a second duct resistance being a resistance of the gas-supply duct on a lumen side with respect to the buffer tank.

8. The endoscope gas-supply system according to claim 7, further comprising
a duct resistance control device which determines whether or not the intraluminal pressure of the lumen is more than a predetermined threshold value, and which controls the duct resistance varying device so as to make the first duct resistance larger than the second duct resistance when the intraluminal pressure of the lumen is more than the predetermined threshold value.

9. The endoscope gas-supply system according to claim 8, wherein the duct resistance control device controls the duct resistance varying device so as to make the first duct resistance smaller than the second duct resistance when the intraluminal pressure of the lumen is equal to or less than the predetermined threshold value.

10. An endoscope gas-supply system which supplies gas from a gas supply source into a lumen of a living body, the endoscope gas-supply system comprising:
a gas-supply duct for supplying gas from the gas supply source into the lumen of the living body;
a pressure regulation device which regulates a pressure of the gas supplied from the gas supply source;
a buffer tank which is provided in the gas-supply duct on a lumen side with respect to the pressure regulation device and which temporarily stores the gas flowing through the gas-supply duct;
a pressure measurement device which measures an internal pressure of the buffer tank;
a setting device which sets a set value of the intraluminal pressure of the lumen; and
a pressure control device which calculates the intraluminal pressure of the lumen from a result of the measurement by the pressure measurement device, and which controls the pressure of the gas regulated by the pressure regulation device such that the intraluminal pressure of the lumen becomes the set value set by the setting device, wherein
a first duct resistance of the gas-supply duct on a gas supply source side with respect to the buffer tank is larger than a second duct resistance of the gas-supply duct on a lumen side with respect to the buffer tank.

11. The endoscope gas-supply system according to claim 10, further comprising:
a duct resistance varying device which can vary the ratio of the first duct resistance to the second duct resistance; and
a duct resistance control device which determines whether or not the intraluminal pressure of the lumen is more than a predetermined threshold value, and which controls the duct resistance varying device so as to make the first duct resistance larger than the second duct resistance when the intraluminal pressure of the lumen is more than the predetermined threshold value.

12. The endoscope gas-supply system according to claim 11, wherein the duct resistance control device controls the duct resistance varying device so as to make the first duct resistance smaller than the second duct resistance when the intraluminal pressure of the lumen is equal to or less than the predetermined threshold value.

13. The endoscope gas-supply system according to claim 11, wherein
the duct resistance varying device includes:
a plurality of ducts with different duct resistances from each other, the plurality of ducts being arranged in parallel with each other and connected to the gas-supply duct; and
a selector valve which can selectively switch among the plurality of ducts so as to make one of the plurality of ducts communicate with the gas-supply duct, and
the duct resistance varying device controls the selector valve to cause a desired one of the plurality of ducts to communicate with the gas-supply duct.

14. The endoscope gas-supply system according to claim 11, wherein:
the duct resistance varying device includes a control valve which can vary the cross-sectional area of the gas-supply duct; and
the duct resistance control device controls the control valve so that the duct resistance of the gas-supply duct becomes a desired value.

15. The endoscope gas-supply system according to claim 11, wherein the duct resistance varying device is provided in the gas-supply duct on a gas supply source side with respect to the buffer tank.

16. The endoscope gas-supply system according to claim 11, wherein the duct resistance varying device is provided in the gas-supply duct on a lumen side with respect to the buffer tank.

17. The endoscope gas-supply system according to claim 10, wherein the pressure regulation device is provided in the gas-supply duct.

18. The endoscope gas-supply system according to claim 10, wherein the buffer tank is larger in volume than the lumen.
